(19) United States
(12) Patent Application Publication
(10) Pub. No.: US 2023/0292795 A1
(43) Pub. Date: Sep. 21, 2023

(54) NEW FAST DISSOLVABLE TABLETS

(71) Applicant: DSM IP ASSETS B.V., HEERLEN (NL)

(72) Inventors: Zhenbo MA, Kaiseraugst (CH); Conroy Clive SALMON, Kaiseraugst (CH); Paulo Henrique SANTOS, Kaiseraugst (CH)

(21) Appl. No.: 18/016,544

(22) PCT Filed: Jul. 13, 2021
(86) PCT No.: PCT/EP2021/069415

§ 371 Date: Jan. 17, 2023

Related U.S. Application Data
(60) Provisional application No. 63/056,150, filed on Jul. 24, 2020.

(30) Foreign Application Priority Data
Jul. 27, 2020 (CH) 00928/20

(51) Int. Cl. A23L 2/40; A23L 2/395; A23L 2/52; A23L 33/15; A23L 33/125; A61K 9/00

(52) U.S. Cl. CPC A23L 2/40; A23L 2/395; A23L 2/52; A23L 33/15; A23L 33/125; A61K 9/0095

(57) ABSTRACT

The present invention relates to a new fast dissolvable tablet comprising physiologically active ingredients, which dissolves or can be dispersed in water (or water based liquids) fast.

The present invention relates to a new formulation (a tablet) comprising physiologically active ingredients, which dissolves or can be dispersed in water (or water based liquids) fast. The tablets can be consumed easily by a human when dissolved.

There are many known formulations for physiologically active ingredients (such as vitamins, minerals, carotenoids etc).

An important and commonly used one is an effervescent tablet (also called carbon tablet).

Effervescent or carbon tablets are tablets which dissolve in water (or water based liquids) and release carbon dioxide. Such tablets are products of compression of component ingredients in the form of powders into a dense mass, which is packaged in blister pack, or with a hermetically sealed package with incorporated desiccant in the cap. To use them, they are dropped into water (or water-based liquids) to make a solution.

Effervescent tablets have some advantages over regular tablets, such as the following:
Pleasant taste compared to regular tablets
Effervescent tablets are popular due to the fact they can be dissolved in a liquid such as water or fruit juice, meaning that they often taste better than regular tablets. Conventional tablets dissolve slowly which can result in reduced absorption rates, effervescent tablets, in contrast, dissolve quickly and completely, meaning you get the full benefit from the ingredients.
Distributed more evenly
Conventional tablets dissolve gradually in the stomach once ingested and can sometimes only partially dissolve which can lead to irritation in some cases. The benefit of effervescent tablets is that they dissolve completely and evenly meaning that localised concentrations of the ingredients cannot occur. This means not only a better taste but also less chance of irritation and a more efficient means of ingesting the ingredients.
Increased liquid intake
Effervescent tablets provide the nutritional benefits intended, but in addition to this they also increase liquid intake. This can be especially beneficial if you are dehydrated or ill and not ingesting as much fluid as usual. Effervescent tablets can be a fantastic way of rehydrating as well as reaping the benefits you are taking the tablets for whether this is a dietary supplement.
Easy alternative to regular tablets
They can be a great alternative for those who may have trouble swallowing either due to illness or age. Older individuals may have difficulty swallowing but need to take medication or supplements on a regular basis and in this respect, effervescent tablets can be a lot easier than having to swallow a tablet.
Simple and easy to measure
Effervescent tablets are easily dissolved into water or a liquid of your choice and then after a while are consistent, well mixed and ready to drink. Traditional tablets or powders, however, need to be measured and stirred in repeatedly to avoid an inconsistent drink with lumpy bits.

All these factors combine to make effervescent tablets a very popular choice for those taking tablets for either dietary supplementation or medicinal reasons.

Besides these advantages, there are also some major disadvantages of effervescent tablets, such as a complex production process (very low relative humidity) and, very often, the need for special packaging materials for achieving a good storage stability.

The primary material used in the manufacture of effervescent tablets is relatively hygroscopic, that is, it absorbs moisture from the air. However, this must be prevented because it will initiate the effervescent reaction. One of the principle strategies used to overcome this problem is a completely closed material handling system during production,

Furthermore, the tablets need to be packed in such a way that it that moisture cannot not harm the tablet during storage before use.

Therefore, the goal of the present invention was to find a dissolvable formulation having all the advantages of a typical and well-known effervescent tablet, but not having its disadvantages.

Therefore, the present invention relates to a fast (water) dissolvable effervescent beverage tablet (DS), which a new kind of an effervescent tablet, which comprises
(a) 1-40 weight-% (wt-%), based on the total weight of the fast dissolvable tablet, of at least one active ingredient, and
(b) 20-95 wt-%, based on the total weight of the fast dissolvable tablet, of at least one diluent, and
(c) 2-50 wt-%, based on the total weight of the fast dissolvable tablet, of at least one disintegrant,
and wherein the content of any carbonate is below 1 wt-%, based on the total weight of the fast dissolvable tablet.

The fast dissolvable tablet according to the present invention is always dissolved in water (or a water based liquid) before its consumption. This means that the tablet is not swallowed as such.

All percentages are always add up to 100 in total (in one embodiment of a tablet according to the present invention).

The dissolution rate of the tablet according to the present invention is excellent. The tablet according to the present invention dissolves in a water-based liquid in similar rate as an effervescent tablet. No additional shaking, stirring or other means are necessary to achieve a complete dissolution in an acceptable time.

The size of the tablet according to the present invention can vary.

The size is more or less the same as a conventional effervescent tablet. The typical and preferred size is usually chosen that the amount of the physiologically active ingredient is enough to cover the daily recommended or any desired amount.

The shape of the tablet according to the present invention is not an essential feature. But usually it has a disc-like shape having a diameter of up to 3 cm (preferably 1.5-2.5 cm) and a thickness of up to 0.8 cm (preferably 0.3-0.6 cm) and it has a weight of up to 5 g (preferably 0.2-5 g).

Therefore the present invention relates to a fast dissolvable tablet DS1, which is fast dissolvable tablet DS, wherein the tablet has a disc-like shape.

Therefore the present invention relates to a fast dissolvable tablet DS1′, which is fast dissolvable tablet DS1, wherein the tablet has a diameter of up to 3 cm (preferably 1.5-2.5 cm) and a thickness of up to 0.8 cm (preferably 0.3-0.6 cm).

Therefore the present invention relates to a fast dissolvable tablet DS2, which is fast dissolvable tablet DS, DS1 or DS1′, wherein the tablet has a weight of up to 5 g (preferably 0.2-5 g).

Usually the tablet according to the present invention is dissolved or dispersed in a glass of water (or water based liquid), which is usually between 0.1-0.4 liter.

The tablets according to the present invention dissolved rapidly. It has similar dissolving properties as a usual effervescent tablets.

The dissolving time is less than 4 minutes (or even less than 2 minutes).

The tablet according to the present invention is soluble in pure water as well as in water-based solvents. This means the tablet according to the present invention can also be dissolved in any water based liquid (such as fruit juices, milk, smoothies, etc). The liquid can be cold or hot. The liquid can be carbonated or non-carbonated.

Preferred embodiments according to the present inventions are fast dissolvable tablets comprises 5-30 wt-%, based on the total weight of the fast dissolvable tablet, of at least one active ingredient.

More preferred embodiments according to the present inventions are fast dissolvable tablets comprises 5-20 wt-%, based on the total weight of the fast dissolvable tablet, of at least one active ingredient.

Especially preferred embodiments according to the present inventions are fast dissolvable tablets comprises 7-15 wt-%, based on the total weight of the fast dissolvable tablet, of at least one active ingredient.

Therefore the present invention relates to a fast dissolvable tablet DS3, which is fast dissolvable tablet DS, DS1, DS1′ or DS2, wherein the tablet comprises 5-30 wt-%, based on the total weight of the fast dissolvable tablet, of at least one active ingredient.

Therefore the present invention relates to a fast dissolvable tablet DS3′, which is fast dissolvable tablet DS, DS1, DS1′ or DS2, wherein the tablet comprises 5-20 wt-%, based on the total weight of the fast dissolvable tablet, of at least one active ingredient.

Therefore the present invention relates to a fast dissolvable tablet DS3″, which is fast dissolvable tablet DS, DS1, DS1′ or DS2, wherein the tablet comprises 7-15 wt-%, based on the total weight of the fast dissolvable tablet, of at least one active ingredient.

The active ingredients used in the tablets according to the present invention are such, which are usually used in common effervescent tablets.

Suitable active ingredients are vitamins, prebiotics, probiotics, carotenoids, minerals and any other dietary ingredient.

Therefore the present invention relates to a fast dissolvable tablet DS4, which is fast dissolvable tablet DS, DS1, DS1′, DS2, DS3, DS3′, or DS3″, wherein the at least active ingredient is chosen from the group consisting of vitamins, prebiotics, probiotics, carotenoids, minerals and any other dietary ingredient.

The active ingredients used in the tablet according to the present invention can be used in pure form as well as in preformulated form (which means that one or more active ingredient is formulated before used in the production of the tablet according to the present invention).

The vitamins according to the present invention can be fat-soluble as well as water-soluble.

The fat-soluble vitamins are selected from the group consisting of vitamin A, D, E and K. These vitamins are usually used as preformulated form in the tablet so that the preformulated form can be dispersed in water.

The water-soluble vitamins are selected from the group consisting of vitamin B1 (thiamine), vitamin B2 (riboflavin), vitamin B3 (niacin, niacinamide), vitamin B5 (pantothenic acid), vitamin B6 (pyridoxine, pyridoxamine, pyridoxal), vitamin B7 (biotin), vitamin B9 (folic acid, folinic acid), vitamin B12 (cyanocobalamin, hydroxycobalamin, methylcobalamin), and vitamin C (ascorbic acid).

The prebiotics are usually any commonly used dietary fibers (such as inulin).

The probiotics are i.e. Lactobacillus or bifido bacterium.

Carotenoids are chosen form the group consisting of beta-carotene, lycopene, lutein, bixin, astaxanthin, apocarotenal, beta-apo-8′-carotenal, beta-apo-12′-carotenal, canthaxanthin, cryptoxanthin, citranaxanthin and zeaxanthin. These carotenoids are usually used as preformulated form in the tablet.

The minerals are chosen from the group consisting of minerals, which are added to the formulation are Sodium, Potassium, Calcium, Iron, Zinc, and Magnesium.

A suitable trace element is for example iodine.

Therefore the present invention relates to a fast dissolvable tablet DS5, which is fast dissolvable tablet DS, DS1, DS1′, DS2, DS3, DS3′, DS3″ or DS4, wherein the at least active ingredient is chosen from the group consisting of vitamins chosen from the group consisting of vitamin A, vitamin D (vitamin D3), vitamin E, vitamin K1, vitamin K2, vitamin B1, vitamin B2, vitamin B3, vitamin B5, vitamin B6, vitamin B7, vitamin B9, vitamin B12 and vitamin C;
and/or from carotenoids chosen from the group consisting of beta-carotene, lycopene, lutein, bixin, astaxanthin, apocarotenal, beta-apo-8′-carotenal, beta-apo-12′-carotenal, canthaxanthin, cryptoxanthin, citranaxanthin and zeaxanthin; and/or from inulin; and/or
from probiotics chosen from the group consisting of Lactobacillus and bifido bacterium; and/or
from minerals chosen from the group consisting of Sodium, Potassium, Calcium, Iron, Zinc, and Magnesium;
and/or from any other dietary ingredient (such as trace elements, plant extract etc).

A preferred embodiment of the present invention is as fast dissolvable tablet comprising 25-90 wt-%, based on the total weight of the fast dissolvable tablet, of at least one diluent.

A more preferred embodiment of the present invention is as fast dissolvable tablet comprising 30-80 wt-%, based on the total weight of the fast dissolvable tablet, of at least one diluent.

Suitable diluents are sugar alcohols such as mannitol, sorbitol and xylitol and disaccharides such as sucrose or lactose.

Therefore the present invention relates to a fast dissolvable tablet DS6, which is fast dissolvable tablet DS, DS1, DS1′, DS2, DS3, DS3′, DS3″, DS4 or DS5, wherein the tablet comprises 25-90 wt-%, based on the total weight of the fast dissolvable tablet, of at least one diluent.

Therefore the present invention relates to a fast dissolvable tablet DS6′, which is fast dissolvable tablet DS6, wherein the tablet comprises 30-80 wt-%, based on the total weight of the fast dissolvable tablet, of at least one diluent.

Suitable diluents are sugar alcohols such as mannitol, sorbitol and xylitol and disaccharides such as sucrose or lactose.

Therefore the present invention relates to a fast dissolvable tablet DS7, which is fast dissolvable tablet DS, DS1, DS1′, DS2, DS3, DS3′, DS3″, DS4, DS5, DS6 or DS6′, wherein the least one diluent is chosen from the group consisting of sugar alcohols (such as mannitol, sorbitol and xylitol) and disaccharides (such as sucrose and lactose).

A preferred embodiment of the present invention is as fast dissolvable tablet comprising 3-45 wt-%, based on the total weight of the fast dissolvable tablet, of at least one disintegrant.

A more preferred embodiment of the present invention is as fast dissolvable tablet comprising 15-40 wt-%, based on the total weight of the fast dissolvable tablet, of at least one disintegrant.

Suitable disintegrant are starches, crospovidone (cross linked polyvinyl N-pyrrolidone), co-processed sugar alcohol with starch and sodium starch glycolate.

Therefore the present invention relates to a fast dissolvable tablet DS8, which is fast dissolvable tablet DS, DS1, DS1′, DS2, DS3, DS3′, DS3″, DS4, DS5, DS6, DS6′ or DS7, the tablet comprises 10-45 wt-%, based on the total weight of the fast dissolvable tablet, of at least one disintegrant.

Therefore the present invention relates to a fast dissolvable tablet DS8′, which is fast dissolvable tablet DS, DS1, DS1′, DS2, DS3, DS3′, DS3″, DS4, DS5, DS6, DS6′ or DS7, the tablet comprises 15-40 wt-%, based on the total weight of the fast dissolvable tablet, of at least one disintegrant.

Therefore the present invention relates to a fast dissolvable tablet DS9, which is fast dissolvable tablet DS, DS1, DS1′, DS2, DS3, DS3′, DS3″, DS4, DS5, DS6, DS6′, DS7, DS8 or DS8′, wherein the at least one disintegrant is chosen from the group consisting of starches, crospovidone (cross linked polyvinyl N-pyrrolidone), co-processed sugar alcohol with starch and sodium starch glycolate.

Furthermore the tablet according to the present invention can comprise further ingredients (auxiliary agents), such as flavours, colours, fillers, lubricants and sweeteners. These ingredients are not essential for the invention, but they are useful to design a tablet, which is useful for the desired aim of the tablet.

Such ingredients can be present in the tablets according to the present invention in amount of up to 20 wt-%, based on the total weight of the tablet.

Usually when they are used they are present in an amount of 2-20 wt-% (preferably 3-15 wt-%), based on the total weight of the tablet.

Therefore the present invention relates to a fast dissolvable tablet DS10, which is fast dissolvable tablet DS, DS1, DS1′, DS2, DS3, DS3′, DS3″, DS4, DS5, DS6, DS6′, DS7, DS8, DS8′ or DS9, wherein the tablet comprises at least one auxiliary agent chosen from the group consisting of, flavours, colours, fillers, lubricants and sweetener.

Therefore the present invention relates to a fast dissolvable tablet DS10′, which is fast dissolvable tablet DS10, wherein the tablet comprises 2-20 wt-% (preferably 3-15 wt-%), based on the total weight of the tablet, of at least one auxiliary agent.

Furthermore a preferred embodiment of the present invention is a tablet, wherein the carbonate content is below 0.5 wt-%, based on the total weight of the tablet.

A more preferred embodiment of the present invention is a tablet, wherein the carbonate content is below 0.3 wt-%, based on the total weight of the tablet.

Especially preferred embodiment of the present invention is a tablet which is essentially free of any carbonate. This means that the tablet does not comprise any carbonate. This means that no carbonate is added to the tablet.

Therefore the present invention relates to a fast dissolvable tablet DS11, which is fast dissolvable tablet DS, DS1, DS1′, DS2, DS3, DS3′, DS3″, DS4, DS5, DS6, DS6′, DS7, DS8, DS8′, DS9, DS10 or DS10′, wherein the tablet comprises less than 0.5 wt-%, based on the total weight of the tablet, of carbonate.

Therefore the present invention relates to a fast dissolvable tablet DS11′, which is fast dissolvable tablet DS, DS1, DS1′, DS2, DS3, DS3′, DS3″, DS4, DS5, DS6, DS6′, DS7, DS8, DS8′, DS9, DS10 or DS10′, wherein the tablet comprises less than 0.3 wt-%, based on the total weight of the tablet, of carbonate.

Therefore the present invention relates to a fast dissolvable tablet DS11″, which is fast dissolvable tablet DS, DS1, DS1′, DS2, DS3, DS3′, DS3″, DS4, DS5, DS6, DS6′, DS7, DS8, DS8′, DS9, DS10 or DS10′, wherein the tablet is essentially free of any carbonate.

The tablets according to the present invention can be produced by using commonly known processes.

Usually the following process steps are used
(i) Mixing all ingredients (except the lubricants) and blend them
(ii) Adding the lubricant(s) and mixing all ingredients
(iii) Pressing the mixture into a tablet form which is wished.

The pressing can be done by commonly known and used equipment, such as a “D” type rotary press.

A suitable and ideal tablet according to the present invention has a hardness of around 5 kp to 20 kp, preferably 6 kp-15 kp.

Therefore the present invention relates to a fast dissolvable tablet DS12, which is fast dissolvable tablet DS, DS1, DS1′, DS2, DS3, DS3′, DS3″, DS4, DS5, DS6, DS6′, DS7, DS8, DS8′, DS9, DS10, DS10′, DS11, DS11′ or DS11″, wherein the tablet is essentially free of any carbonate.

The tablet (depending on the choice of active ingredients) can be used as dietary supplements.

The tablets can be packed and sold in any commonly used container for tablet, due to the good storage stability.

The tablet according to the present invention is dissolved or dispersed in water or a water based liquid before it is consumed.

The following examples serve to illustrate the invention.

### EXAMPLES

### Example 1

A tablet of the following composition (table 1) has been produced:

TABLE 1

| Ingredient | mg/tablet | % w/w |
|---|---|---|
| Niacinamide FCC | 23.84 | 0.68 |
| Calcium Pantothenate | 25.93 | 0.74 |
| Pyridoxine Hydrochloride | 2.87 | 0.08 |
| Maltodextrin | 7.36 | 0.21 |
| VC Ascorbic Acid regular | 99.00 | 2.83 |
| Potassium Citrate | 204.25 | 5.84 |
| Dry Vitamin E 50% CWS/S | 79.02 | 2.26 |
| Beta-Carotene 20% S | 10.00 | 0.29 |
| Citric Acid | 93.00 | 2.66 |
| Malic Acid | 28.00 | 0.80 |
| Orange flavor | 80.00 | 2.29 |
| Reb M | 80.00 | 2.29 |
| Pearlitol Flash (co-processed Mannitol with starch) | 1164.52 | 33.27 |
| Pearitol 200 SD (Mannitol) | 1542.21 | 44.06 |
| PEG 6000 | 60.00 | 1.71 |

The tablet with the composition of table 1 has been produced by the following procedure:
1. Sieve all ingredients through a 20 mesh screen.
2. Mix all ingredients except the lubricant in a V shaped blender for 10 mins.
3. Add sieved lubricant into step 2 and mix for 5 mins.
4. Produce tablets on a “D” type rotary press equipped with 0.875″ flat faced and bevel edged toolings.

Compression force 6000 Lbs, Press speed: 55 rpm, Tablet hardness ˜7 kp

This tablet has been dropped in about 236 ml (8 oz) of water and was shaken gently. The tablet was dissolved rapidly and completely within 2 mins.

1. A fast dissolvable-effervescent beverage tablet, which comprises
(a) 1-40 weight-% (wt-%), based on the total weight of the fast dissolvable tablet, of at least one active ingredient, and
(b) 20-95 wt-%, based on the total weight of the fast dissolvable tablet, of at least one diluent, and
(c) 2-50 wt-%, based on the total weight of the fast dissolvable tablet, of at least one disintegrant,
and wherein the content of any carbonate is below 1 wt-%, based on the total weight of the fast dissolvable tablet.

2. Tablet according to claim 1, wherein the tablet has a disc-like shape.

3. Tablet according to claim 1 comprising 5-30 wt-%, based on the total weight of the fast dissolvable tablet, of at least one active ingredient.

4. Tablet according to claim 1, wherein the at least active ingredient is chosen from the group consisting of vitamins, prebiotics, probiotics, carotenoids, minerals and any other dietary ingredient.

5. Tablet according to claim 1 comprising 25-90 wt-%, based on the total weight of the fast dissolvable tablet, of at least one diluent.

6. Tablet according to claim 1, wherein the least one diluent is chosen from the group consisting of sugar alcohols (such as mannitol, sorbitol and xylitol) and disaccharides (such as sucrose and lactose).

7. Tablet according to claim 1 comprising 10-45 wt-%, based on the total weight of the fast dissolvable tablet, of at least one disintegrant.

8. Tablet according to claim 1, wherein the at least one disintegrant is chosen from the group consisting of starches, crospovidone (cross linked polyvinyl N-pyrrolidone), co-processed sugar alcohol with starch and sodium starch glycolate.

9. Tablet according to claim 1, wherein the tablet comprises at least one auxiliary agent chosen from the group consisting of, flavours, colours, fillers, lubricants and sweetener.

10. Tablet according to claim 1, wherein the carbonate content is below 0.5 wt-%, based on the total weight of the tablet.

11. Tablet according to claim 1, wherein the tablet which is essentially free of any carbonate.

12. Tablet according to claim 1, wherein the tablet has a hardness of about 5 to 20 kp.
